(19) United States
(12) Patent Application Publication
(10) Pub. No.: US 2013/0267871 A1
(43) Pub. Date: Oct. 10, 2013

(54) APPARATUS, SYSTEM AND METHOD OF MONITORING BODILY FLUID OUTPUT IN A HEALTHCARE ENVIRONMENT

(71) Applicants: Barbara Delaney, Philadelphia, PA (US); Rodeen Rahbar, Alexandria, VA (US); Miro Uchal, Jacksonville, FL (US)

(72) Inventors: Barbara Delaney, Philadelphia, PA (US); Rodeen Rahbar, Alexandria, VA (US); Miro Uchal, Jacksonville, FL (US)

(73) Assignee: RUD, LLC, Waverly, PA (US)

(21) Appl. No.: 13/802,493

(22) Filed: Mar. 13, 2013

Related U.S. Application Data
(60) Provisional application No. 61/610,284, filed on Mar. 13, 2012.

(51) Int. Cl. A61B 5/20; A61B 5/00

(52) U.S. Cl. CPC A61B 5/208; A61B 5/746; A61B 5/742
USPC 600/584

(57) ABSTRACT

An apparatus, system and method of monitoring fluid output are disclosed. The apparatus, system and method includes at least one exit route for the fluid output, a collection point for the fluid output, and at least one output reader for display of at least one output reading device. The output reader includes a detector, such as a weight sensor, an ultraviolet sensor, a float sensor, a mechanical diaphragm/counter, or an electromechanical or electrical sensor, through which the fluid output passes, at least one local memory at which a counter output of the detector is stored, and a buffer that forwards the counter output accumulated at the at least one local memory to at least one remote memory at a predetermined interval.

### CROSS-REFERENCE TO RELATED APPLICATIONS

This application claims the benefit of U.S. Provisional Application Ser. No. 61/610,284, filed Mar. 13, 2012, the entire disclosure of which is incorporated by reference herein as if set forth in its entirety.

### BACKGROUND OF THE INVENTION

a. Field of the Invention

The instant invention relates to medical devices for monitoring one or more patients, and more specifically, to an apparatus, system and method of monitoring bodily fluid output in a healthcare environment.

b. Description of the Background

With respect to a variety of different medical conditions and treatments, it is vital that a patient's urine output be monitored. In typical embodiments of urine monitoring, a patient outputs urine, via any of a variety of known methods, into an output bag, or “leg bag.”

For example, traditional leg bags drain body fluids, such as urine, via gravity. These leg bags are then typically emptied into a graduated cylinder to accurately record the amount of output. Needless to say, the amount of urine upon emptying of the bag, as well as the precise timing of the emptying of the bag, must be very carefully recorded in order to provide useful, health-related information for the subject patient. This process is labor intensive and susceptible to human error. For example, a misrepresentation of the time of emptying a bag by as little as 15 minutes to 1 hour may constitute an error sufficient to cause an unhealthy patient to be deemed healthy by a monitoring physician. However, it would be advantageous to provide an improved system that made use, for example, of such disposable leg bags, due to the low cost associated therewith. For example, wholesale pricing of legs bags is a low as $1.86/disposable drainage bags when purchased in lots of 20-100.

Therefore, physicians and health care providers have a need for an inexpensive, automated recording and system of alarms of the output of body fluids, in particular urine.

### SUMMARY OF THE INVENTION

The present invention is and includes an apparatus, system and method of monitoring urine output. The apparatus, system and method includes at least one exit route for the urine, a collection point for the urine, and at least one output reader for display of at least one output reading device. The output reader includes a detector, such as a weight sensor, an ultraviolet sensor, a float sensor, a mechanical diaphragm/counter, or an electromechanical or electrical sensor, through which the urine passes, at least one local memory at which a counter output of the detector is stored, and a buffer that forwards the counter output accumulated at the at least one local memory to at least one remote memory at a predetermined interval.

Thus, the present invention may provide an inexpensive, automated recording of the output of body fluids as discussed herein, and particularly, by way of example, of the output of urine

### BRIEF DESCRIPTION OF THE DRAWINGS

The present invention will be described in conjunction with the following figures, wherein like numerals denote like aspects of the invention, and wherein:

FIG. 1 is a block diagram illustrating the aspects of the present invention.

FIG. 2 is an exemplary embodiment of the present invention.

FIG. 3 is an exemplary embodiment of the present invention.

FIG. 4 is an exemplary embodiment of the present invention.

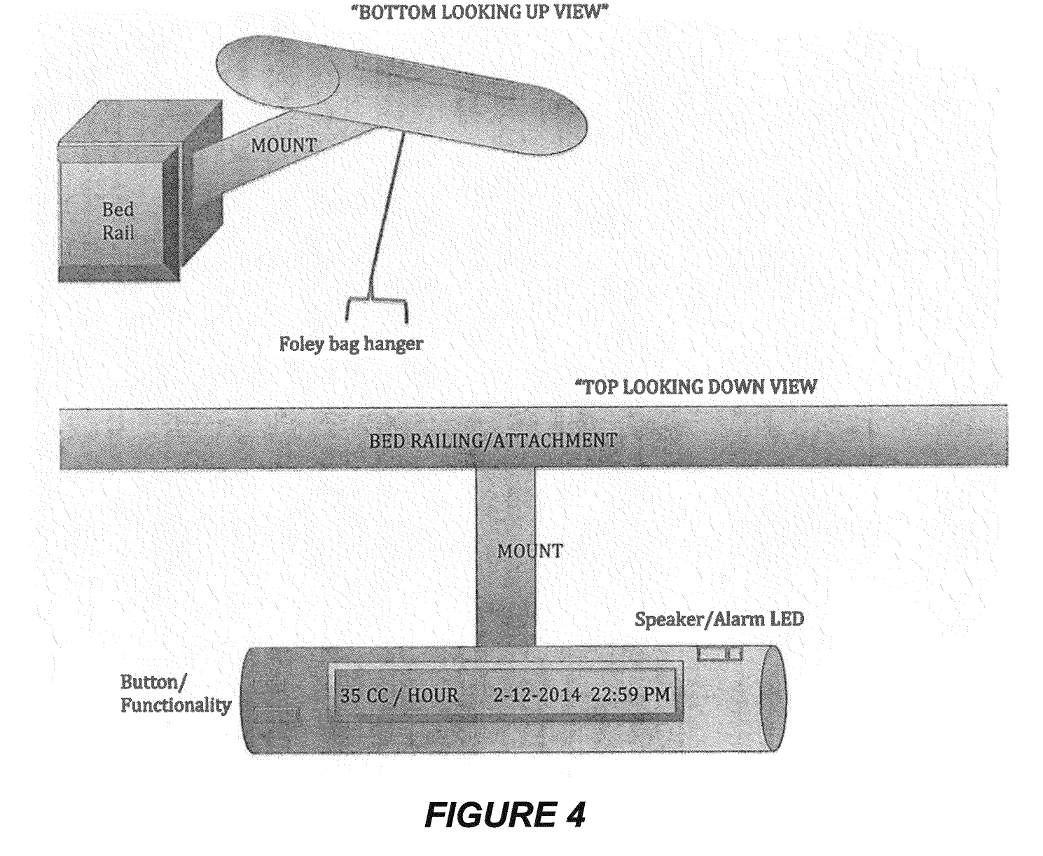

### DETAILED DESCRIPTION OF THE INVENTION

It is to be understood that the figures and descriptions of the present invention have been simplified to illustrate elements that are relevant for a clear understanding of the present invention, while eliminating, for the purpose of clarity, many other elements found in typical medical devices, systems and methods. Those of ordinary skill in the art will recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art. Furthermore, the embodiments identified and illustrated herein are for exemplary purposes only, and are not meant to be exclusive or limited in their description of the present invention.

In certain exemplary embodiments, it is desired to provide a cost-effective determination of average fluid output, such as per half hour or hour. Humans usually make about 30 cc of urine per hour on average, although it is possible to make urine at rates of 10 times that average, such as up to 300-500 cc per hour. Typically, it is not necessary to measure real time flow (cc/second, minute, etc.), but only necessary to measure average low over a predetermined time frame.

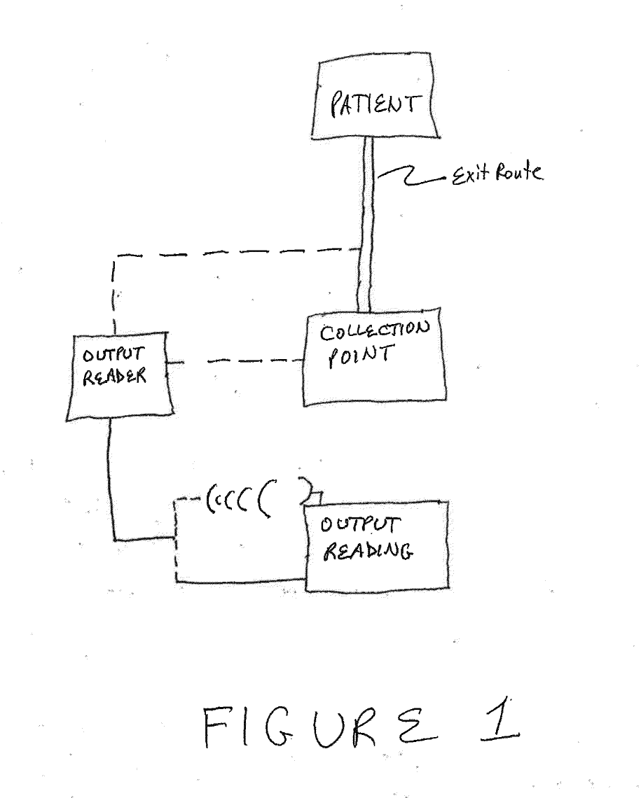

Referring now to FIG. 1, there is shown a system of monitoring urine output in accordance with the present invention. In the illustrated embodiment, the system includes an exit route for urine or other bodily fluid(s) (hereinafter “urine”), a collection point for urine, and an output reading device. Further, the system includes a urine output reader, a reading from which is forwarded to the aforementioned output reading device, such as a memory or output device, continuously, or at predetermined intervals, such as quarter-hourly, hourly, or the like. The system of the present invention may thereby enable the triggering of an alarm or similar notification at a local and/or remote monitor when low to nil volume of flow over the predetermined interval occurs. This safety feature is similar to cardiac monitoring, and serve as the only or principal early sign of clinical status deterioration, system disengagement or malfunction.

In typical embodiments, the exit route would include one or more tubes associated with, for example, a catheter or the like within, or otherwise associated with, the subject patient's body. The composition of such tubes will be apparent to those skilled in the art, and will typically include plastics, polymers, glass, woven materials, or the like.

The collection point for urine may, for association with the embodiments described hereinbelow, be any one or more collection points known to those skilled in the art, including, but not limited to, a cylinder, bag, box, or the like, formed of flexible or inflexible material dependent upon the selected embodiment discussed hereinafter, and formed of, for example, water-proof materials such as plastics, polymers, glass, woven materials, or the like.

The output reading device of the system may, in certain exemplary embodiments, constitute the only non-disposable, and/or non-sterile, portion of the system. As such, the output reading device may constitute the lone portion of the system having associated therewith any appreciable expense and/or maintenance costs. The output reading device may be stationary, or portable, such as handheld, and may be local, wired, or the like to the output reader, and/or may be remote from the output reader, such as via a WAN, LAN, or like wireless network, a cellular network, a blue tooth network, or the like.

In an exemplary embodiment, a handheld output reading device may be hand-held, battery driven, and rechargeable computer, and may have associated therewith one or more microprocessors for manipulating one or more software programs. Likewise, a stationary or portable, non-handheld reading device may have associated therewith such one or more software programs. Further, in certain embodiments, stationary, portable non-handheld, and handheld out reading devices may be operable in a single system or network, such as within the same hospital environment.

Such one or more software programs may include, for example, a graphical user interface (a “GUI”), wherein the GUI provides information of use in making decisions regarding the health and/or treatment of one or more patients. For example, the software may process one or more radio frequency (“RE”) signals received from the output reader, such as via one or more wired or wireless “hubs” associated with the one or more output readers, and the software may then manipulate the received RF signal into a reading of urinary output for presentation, via the GUI, to one or more users. As such, the software may record and present, via the GUI, the time of measurement and the measurement, and may track and/or display all measurements of all output readers. Thereby, trends in urine output over time may be tracked, displayed and/or manipulated.

The GUI may additionally include, for association with the urinary output reading device, software for presenting, or locally or remotely accessing, indications stemming from the subject urinary output reading device. Similarly, the GUI may provide one or more instructions to a user for measurement, such as to make sure that, to the extent the output reader constitutes a weight measurement, the collection point is upright and not in contact with, resting on, or being pressed by, the patient, the ground any equipment, or other elements in a typical care-giving environment.

In certain embodiments, a user of the GUI may request a measurement from one or more patients within a given system. Similarly, measurements may be “pushed” to the GUI, continuously or at predetermined intervals. Further, intervals may be variable, such as based on patient, patient location, previously recorded urinary output by patient, or patient condition, for example.

The output reading device, in conjunction with the output reader, of the present invention may be expandable to, for example, other types of fluid outputs. For example, the system of the present invention may be employed with IV poles, chest tube drainage systems, Blake drains, and like drainage systems.

The output reader may take any number of forms, and may most preferably be present in association with one of the exit route or the collection point. For example, within the exit route may be placed a mechanical, electromechanical, or electrical flow meter. Such a flow meter may constitute, for example, a diaphragm calibrated to “click” upon the passing of a predetermined volume, and thereby a number of clicks per unit time would equate to the passing of a particular volume through the exit route in that particular time. The conversion to volume passed may be performed by the output reader, or may be performed at the output reading device discussed hereinabove. Additionally and alternatively, those skilled in the art will appreciate that such metering may be performed through the use of electrical sensors placed about the circumference of the exit route, such as ultraviolet sensors, dielectric or conductivity sensors (which may necessitate the passing of a small electrical charge through, and the imparting of a low level of conductivity to, the exit route), and the like.

Further, the output reader may be placed at the reference point, as discussed hereinabove. In such an embodiment, variations in the weight, volume, or conductivity at the collection point may be made per unit time. For example, a spring tensioner may be provided from which a leg bag may be suspended, with variations in the tension providing an indication of variation in the weight of the hanging bag. Needless to say, the weight may be converted to volume based on the known characteristics of urine, thereby, again, providing a measure of volume of urine per unit time. For example the software may use the average specific gravity of urine to convert weight to volume. Because of the small variability in the actual specific gravity of urine, an error, such as +/−2%, may occur in converting weight to volume. Those skilled in the art will appreciate that, similarly, a force based transducer system may be employed.

Additionally, a “float” sensor, such as those used in gasoline tanks, may be employed to provide an indication of volume at the collection point at any given time. Further, a sensor array, such as an infrared or ultraviolet array, may detect variations in the urine volume at the collection point over time. Yet further, aspects of the collection point may make available low levels of conductivity and, upon passing a low level signal through the bag, variations in electrical characteristics, such as conductivity, resistivity, dielectric value, and the like, may be converted to a value of urine volume. Yet further, an inverted, or reversed, IV-type system may be employed.

Needless to say, in certain of the aforementioned embodiments, one or more batteries, or like voltage sources, may be provided, such as in association with each bag, or generally by each bedside, for example. Similarly, the system of the present invention may draw necessary power from the output reading device, such as a handheld reader, or from the potential of, for example, the volume flow itself. Further, any or all of the aforementioned methodologies may be associated with at least one means for passing an indicating signal, such as via RF or via wired current. Such means include, but are not limited to, wires associated with the one or more sensors, a broadcast RF signal generator for association with an RF hub, a blue tooth or infrared broadcast signal generator, or the like. It almost goes without saying that information may be stored locally, such as for batch download via any of the aforementioned methodologies, for example. Further, of course, information may be stored more broadly locally, such as within a hospital, for a predetermined time frame, such as for the duration of a particular patient's stay, or for 30 days, prior to download via a network to, for example, a central repository of such information.

In certain preferred embodiments, the information or signal broadcast via wired or wireless communication indicative of the urine volume may additionally include a broadcast of an identifier of the collection point, and/or of the patient associated with the collection point. Such a broadcast may be performed by, for example, an RF tag, tagless RF, or a bar code, for example, associated with a bag, with a patient wrist band, with a bed, or the like. Further, at times, a collection point may need to be changed, such as if the patient develops a bladder infection. As such, the system of the present invention may readily link previous data to new data, such as may be associated with a new collection point but for the same patient.

In conjunction with other components of embodiments of the present disclosure, the output reading device may be configured to perform other tasks as well. For example, the reading may act as a clearinghouse for information about various aspects of fluid management in addition to mere urine output. For example, the output reading device may be configured to quantify a time of patient urinary catheterization. Specifically, upon a urine bag initially being connected to a patient, the output reader may trigger the output reading to start a timer to clock a length of time any urine bag is connected to the patient. By clocking this time, embodiments of the disclosure are able to aid in a hospitals' compliance with certain CMS guidelines.

Specifically, in an effort to prevent, or at least decrease a susceptibility to urinary tract infection, the CMS guidelines may set a maximum duration of time any one urine bag may be connected to a patient. This maximum amount of time, or any other target connectivity time may be entered (through the GUI discussed herein) by a user (e.g., a nurse) of the output reading device. As such, upon the clocked duration reaching this maximum set time, an alarm mechanism (programmed or otherwise connected to the output reading) may be configured to alert any staff to discontinue use of the currently connected urine bag and to replace with a fresh urine bag.

Not only can a design quantify lengths of time urine bags are connected to a patient, embodiments of the present disclosure may also quantify lengths of time central lines (i.e., tubes used to administer intravenous (IV) fluids) are attached to the patient. In a similar fashion to the quantification of length of patient urinary catheterization, upon a central line initially being connected to a patient, the output reader may trigger the output reading to start a timer to clock a length of time that the central line is connected.

In an effort to prevent, or at least decrease a susceptibility to infection by any one of the central lines connected to a patient, guidelines, similar to those set for maximum urine bag connection times, may be instituted. These guidelines may be specific to the type and location of the administered central line. As such, upon connectivity, these parameters, in addition to any suggested maximum time duration, may be set by a user of the system (e.g., output reading). Accordingly, upon the clocked duration reaching this maximum set time, an alarm mechanism (programmed or otherwise connected to the output reading) may be configured to alert any staff to discontinue use of the currently connected central line tube so as to replace with a fresh tube.

Besides measuring a length of time, the afore-discussed central lines and bags may be connected to the patient, embodiments of the present disclosure may also measure an amount and type of fluid administered to the patient, which can be effected by sensors and meters of the types discussed hereinthroughout. Similarly these respective sensors and meters may be attached to their respective tubes in a similar configuration as those attached to the reference point of the respective tube.

As discussed above, any data relating to the patient or any type, location, and quantification of fluids administered to, and potentially drained from, the patient may be entered manually by a user of the system (e.g., nurse). However, sensors may be added to automate entry of any patient data as well. Those skilled in the art will appreciate that in light of the disclosure hereinthroughout, the techniques discussed may be applied to other types of fluid drains in addition to urine, which may include, but not limited to abdominal fluids, intracranial, and thoracic.

To aid in the management of any of the aforementioned aspects of fluid management, embodiments of the present disclosure may include a disposable patient wristband that will interface with the output reading device and/or output reader. Specifically, the wristband may facilitate the transfer or communication of data to other patient rooms or environments internal to the hospital, and or potentially other medical institutions. For example, the wristband may have a universal serial bus (USB) type clasp communicatively connected to the output reading of a system according to an embodiment to store back up patient fluid management data related to hospital admission. This fluid management data may be captured by the patient wristband and, by its attachment and direct association to a particular patient, the fluid management data may be easily provided to other health care professionals involved in the care of the patient. Due to the confidential nature of any patient data, the wristband may also be configured to encrypt the data, so as to only allow access by authorized health care professionals. Further, upon discharge of the patient, the wristband may be disposed above.

Those skilled in the art will appreciate that, in light of the disclosure, the output reading may interface with other medical record systems and electronic health records through a USB attached to the wristband. Further, the wristband may be configured to allow for communication/interfacing with the aforementioned records through any of the communication means discussed herein. It is also important to note that medical records standards as known in the art may be used in formatting the patient data discussed herein. As such, according to embodiments of the present disclosure, the system may be configured to be compatible with “CAPSULE”, a service that allows medical devices to communicate with the various electronic health records systems available to medical institutions.

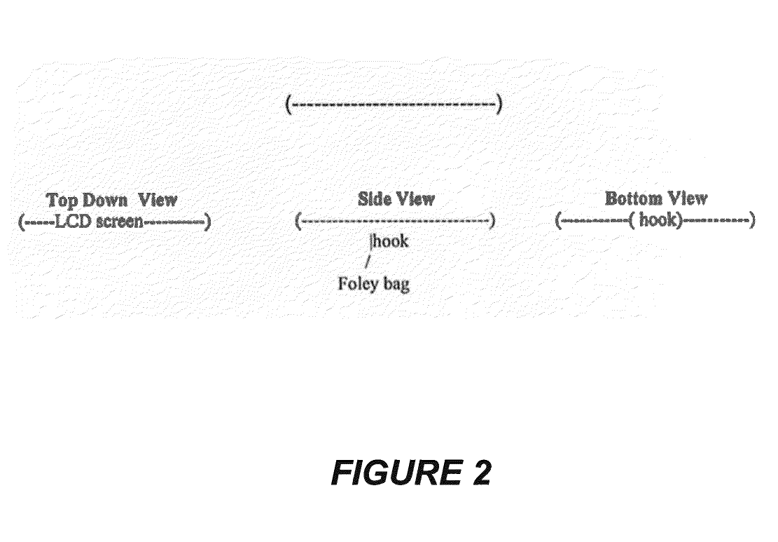

In an alternative embodiment, the afore-discussed flow meter device may be shaped as a two foot long cylinder (with maybe a 2-3 inch diameter) that would lock and/or unlock into a hospital bed mount. The cylinder shape may be represented as in FIG. 2.

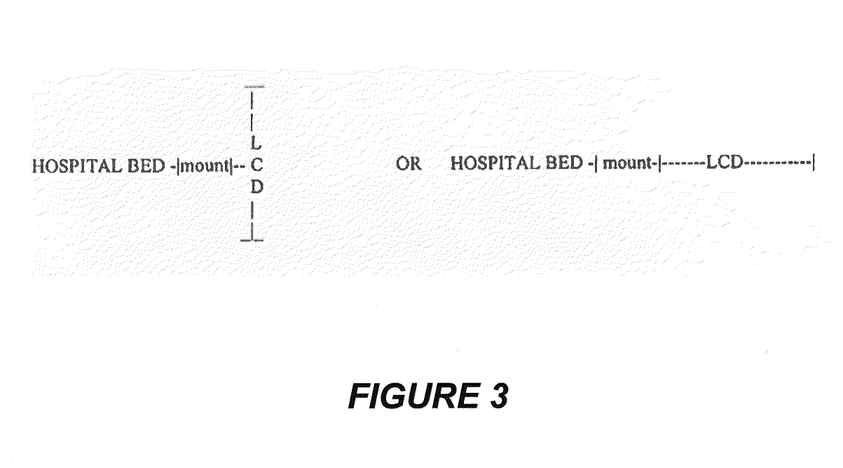

The cylinder flow meter device would be durable in case things bang into it, such as stainless steel or a high quality plastic, with functionality on the top view. A locking system for a mount (for example, a screw or ball in socket) may be included so that it can lock into the mount at the foot of a bed. The mount should allow the device to be swung out of the way. It may connect to the mount either straight out or at its middle (e.g., T-shaped) depending on what is easier for the nursing staff. A top down configuration may be illustrated as shown in FIG. 3.

The aforediscussed smart flow meter may act as both a brain and an attachment point for disposables in the system. On a top view, it would be button functionality, LCD, and ALARM indicators. On a side view, it would be polished stainless steel or plastic. On a bottom view, it may be a weighing mechanism “hook” shaped to hand foley bags.

As mentioned above, embodiments of the present disclosure may also contain disposable “RF” ID, NFC or other communications ID type tags. These tags may be used to identify each bag as unique. The smart flow device may assign data to the tag it is currently hanging. It may be configured to store large amounts of data (e.g., at least weeks worth of data) on tag using an active RF technology.

In light of the discussion above, embodiments of the present disclosure may keep an accurate track of a date, time, and display on a display screen. Embodiments may measure the weight of the hanging foley bags, and use a conversion factor to estimate the volume of the foley bags at time points (e.g, every 30 minutes). Embodiments may clearly display on an LED/LCD screen an average output over hours and/or totals per day, upon user request.

Several different types of alarms may be employed. For example, one type may notify staff when a volume estimate in the bag is under a critical threshold (for example, less than 30 cc in one hour). There may be a default critical amount but this may be customizable in menu options.

Another alarm may notify staff when a foley bag has been connected to a patient for 30 hours after a surgical procedure. This may apply to RF tags pre-manufactured as post-surgical.

And yet, another alarm may notify staff when a foley bag may be reaching capacity and, consequently, may need to be emptied.

It should be appreciated, that in light of the disclosure herein, that the LED (or LCD) screen may include scrolling text that communicates key parameters based on user input (for example, hourly/daily urine output estimates, etc).

Those of ordinary skill in the art will recognize that many modifications and variations of the present invention may be implemented without departing from the spirit or scope of the invention. Thus, it is intended that the present invention cover the modification and variations of this invention provided they come within the scope of the appended claims and their equivalents.

What is claimed is:

1. A system of monitoring fluid output, comprising:
at least one exit route for the fluid output;
a collection point for the fluid output; and
at least one output reader for display on at least one output reading device, comprising:
a detector past which the fluid output passes;
at least one local memory at which an output of the detector is stored;
a buffer that forwards the output at the at least one local memory to at least one remote memory at a predetermined interval;

2. The system of claim 1, wherein the at least one output reading device comprises an alarm device configured to send an alarm signal based on fluid management conditions.

3. The system of claim 2, wherein the fluid management conditions comprise the output reaching a set maximum duration of time that the collection point is attached to a patient.

4. The system of claim 2, wherein the at least one exit route comprises a central line tube.

5. The system of claim 4, wherein the fluid management conditions comprise the output reaching a set maximum duration of time that the central line tube is attached to a patient.

6. The system of claim 5, wherein the maximum duration of time is predefined.

7. The system of claim 1, wherein the at least one output reading device is communicatively coupled to a wristband attached to the patient.

8. The system of claim 1, wherein the detector comprises an ultraviolet sensor.

9. The system of claim 1, wherein the detector comprises a mechanical diaphragm.

10. A method of monitoring fluid, comprising:
routing the fluid through at least one exit route;
collecting the fluid at a collection point; and
detecting, by at least one detector, characteristics of the fluid to transmit fluid output;
storing, at at least one local memory the fluid output;
forwarding the fluid output to at least one remote memory at a predetermined interval;

11. The method of claim 10, further comprising:
transmitting an alarm based on fluid management conditions.

12. The method of claim 11, wherein the fluid management conditions comprise the fluid output reaching a set maximum duration of time that the collection point is attached to a patient.

13. The method of claim 11, wherein the at least one exit route comprises a central line tube.

14. The method of claim 13, wherein the fluid management conditions comprise the fluid output reaching a set maximum duration of time that the central line tube is attached to a patient.

15. The method of claim 14, wherein the maximum duration of time is predefined.

16. The method of claim 10, wherein the at least one detector comprises an ultraviolet sensor.

17. The method of claim 10, wherein the at least one detector comprises a mechanical diaphragm.
